(19) United States
(12) Patent Application Publication
(10) Pub. No.: US 2008/0145319 A1
(43) Pub. Date: Jun. 19, 2008

(54) Compositions Comprising Extracts from Sanguinaria or Macleaya

(76) Inventors: Ezio Bombardelli, Groppello Cairoli (IT); Massimo Ronchi, Milano (IT); Paola Di Nicolo, Milano (IT)

Correspondence Address: JONES DAY, 222 EAST 41ST ST, NEW YORK, NY, 10017, US

(21) Appl. No.: 11/793,089

(22) PCT Filed: Dec. 6, 2005
(86) PCT No.: PCT/EP2005/013047

§ 371 Date: Jan. 31, 2008

(30) Foreign Application Priority Data
Dec. 17, 2004 (IT) MI2004A002414

(51) Int. Cl. A61K 9/68; A61K 36/66; A61K 36/45; A61P 31/04

(52) U.S. Cl. 424/48; 424/749; 424/732

(57) ABSTRACT

Compositions containing: 1. Anthocyanosides, and/or 2. Procyanidins, 3. Isoquinoline alkaloids extracted from Sanguinaria canadensis, Macleaya cordata or Macleaya microcarpa, and optionally 4. a Zanthoxylum bungeanum and/or Echinacea angustifolia lipophilic extract.

The present invention relates to novel compositions containing anthocyanosides and/or procyanidins in combination with bactericidal isoquinoline alkaloids and optionally with a lipophilic extract of Zanthoxylum bungeanum and/or Echinacea angustifolia.

### TECHNOLOGICAL BACKGROUND

Throat redness and inflammation with formation of plaques are a frequent aspect of common influenza, cold and other cold diseases. Common cold and influenza, which affect on the average up to three times a year both children and adults, are related to mild viral infections caused by rhinovirus (40%), coronavirus (10%) and to a less extent adenovirus and parainfluenza virus. Although no specific treatments for these pathologies exist, antihistamines and decongestants are considered useful, as the oedema reduction alleviates pain and shortens the duration of the disease underlying inflammation. Said conditions can sometimes involve complications due to secondary bacterial infections, as nasal sinuses are often obstructed by the congestion of mucous membranes wherein pathogenic germs can easily proliferate. In this event, it is necessary to undergo antibiotic treatment.

### DISCLOSURE OF THE INVENTION

The present invention relates to novel compositions containing
a. Anthocyanosides, and/or
b. Procyanidins,
c. Isoquinoline alkaloids extracted from Sanguinaria canadensis, Macleaya cordata or Macleaya microcarpa,
   and optionally
d. a lipophilic extract of Zanthoxylum bungeanum and/or of Echinacea angustifolia.

According to the present invention, “anthocyanosides” comprises both anthocyanosides per se and their aglycones (anthocyanidins).

Anthocyanosides preferably derive from extracts of Vaccinium myrtillus. The extract of bilberry (Vaccinium myrtillus) is known to have marked, particularly topical, anti-inflammatory activity, thanks to its action on capillary permeability and fragility. The preparation of anthocyanosides-containing bilberry extracts is known. Moreover, both bilberry anthocyanosides and procyanindins have bacteriostatic action which prevents bacterial and fungal adhesion, for example at the dental, paradental and mucous membrane levels.

According to the present invention, procyanindins preferably derive from Vitis vinifera extracts, obtained as described in GB 1541469 or from Camellia sinensis extracts, as disclosed in EP 0814823, or other plants containing them, preferably edible plants.

The content in anthocyanosides- and/or procyanosides-containing extracts can range from 20 to 80 mg per unit dose.

According to the present invention, “isoquinoline alkaloids” mean isoquinoline alkaloids having antibacterial activity, particularly sanguinarines and cheleritrines, preferably obtainable from water-alcoholic extracts of Sanguinaria canadensis, Macleaya cordata or Macleaya microcarpa. They have broad-spectrum antimicrobial activity. 0.2% Water-alcoholic solutions have shown therapeutic activity in a number of conditions of the upper respiratory tract, acting on pathogenic gram-positive and gram-negative bacteria, mycetes, fungi and protozoa. The extracts containing these alkaloids have shown strong anti-inflammatory topical activity. The extracts containing the isoquinoline alkaloids are typically present in amounts from 2 to 20 mg per unit dose.

Preferably, the compositions of the invention also comprise lipophilic extracts of Zanthoxylum bungeanum and/or Echinacea angustifolia enriched in isobutylamides. Said extracts have useful analgesic topical activity through inhibition of nervous conduction. Each one of said extracts can be added to the compositions of the invention in amounts ranging from 0.02 to 0.05 mg per unit dose.

Therefore, according to a preferred aspect, the compositions of the invention will contain, in addition to components 1 and/or 2, and 3 indicated above, also an isobutylamide-enriched lipophilic extract of Zanthoxylum bungeanum and/or Echinacea angustifolia. The Zanthoxylum bungeanum extract can be prepared e.g. according to WO 00/02570. The Echinacea extract can be prepared e.g. according to EP 0 464 298. The compositions of the invention are capable of preventing the formation of purulent plaques infected with various saprophytes of the oral cavity, thus avoiding the use of antibiotics, while reducing the progress of the infection. In particular, the compositions of the invention proved to exert a synergistic effect mainly on the duration of the condition. Furthermore, the compositions of the invention are capable of exerting favourable actions, cleaning the oral cavity and removing dental plaque, thanks to the bacterial adhesiveness-reducing effect, as already mentioned, of both bilberry extracts and procyanindins, and to the high activity of isoquinoline alkaloids on anaerobic bacterial strains.

The pharmaceutical compositions will preferably be formulated as tablets for the slow dissolution in the oral cavity or as chewable forms, particularly gums, which provide the slow release of the active principles. These compositions are used in both prophylactic and curative treatments, as well as for the hygiene of the oral cavity.

Therefore, the present invention also relates to the use of
a. Anthocyanosides, and/or
b. Procyanidins,
c. Isoquinoline alkaloids extracted from Sanguinaria canadensis, Macleaya cordata or Macleaya microcarpa,
   and optionally
d. Zanthoxylum bungeanum and/or Echinacea angustifolia lipophilic extract,

for the preparation of compositions for the prevention and the treatment of bacterial infections of the oral cavity and for the hygiene of the oral cavity.

According to a preferred aspect, the compositions of the present invention will also contain essential oils.

Said compositions will be prepared according to well-known conventional methods, such as those described in “Remington's Pharmaceutical Handbook”, Mack Publishing Co., N.Y., USA, together with suitable excipients.

The following examples illustrate the invention in detail.

### EXAMPLE I

### 1000 mg Chewable Tablets Containing

| | | | |
|---|---|---|---|
| 1. | Vaccinium myrtillus alcoholic extract | 40 | mg |
| 2. | Sanguinaria canadensis alcoholic extract | 2 | mg |
| 3. | Zanthoxylum bungeanum extracted purified | 0.025 | mg |
| 4. | Soy lecithin | 30 | mg |
| 5. | Anhydrous citric acid | 5 | mg |
| 6. | L-Cysteine | 5 | mg |
| 7. | Lactose | 200 | mg |
| 8. | Mannitol | 552.475 | mg |
| 9. | Methylcellulose | 40 | mg |
| 10. | Glycerol palmitostearate | 50 | mg |
| 11. | Berry flavour | 40 | mg |
| 12. | potassium acesulfame | 0.5 | mg |
| 13. | Talc | 10 | mg |
| 14. | Sodium bicarbonate | 25 | mg |

### EXAMPLE II

### 1000 mg Chewable Tablets Containing

| | | | |
|---|---|---|---|
| 1. | Vitis vinifera alcoholic extract | 80 | mg |
| 2. | Sanguinaria canadensis alcoholic extract | 2 | mg |
| 3. | Zanthoxylum bungeanum extracted purified | 0.025 | mg |
| 4. | Soy lecithin | 30 | mg |
| 5. | Anhydrous citric acid | 5 | mg |
| 6. | L-Cysteine | 5 | mg |
| 7. | Lactose | 200 | mg |
| 8. | Mannitol | 512.475 | mg |
| 9. | Methylcellulose | 40 | mg |
| 10. | Glycerol palmitostearate | 50 | mg |
| 11. | Berry flavours | 40 | mg |
| 12. | Potassium acesulfame | 0.5 | mg |
| 13. | Talc | 10 | mg |
| 14. | Sodium bicarbonate | 25 | mg |

### EXAMPLE III

### 1000 mg Chewable Tablets Containing

| | | | |
|---|---|---|---|
| 1. | Vaccinium myrtillus alcoholic extract | 40 | mg |
| 2. | Sanguinaria canadensis alcoholic extract | 2 | mg |
| 3. | Echinacea angustifolia lipophylic extract | 5 | mg |
| 4. | Soy lecithin | 30 | mg |
| 5. | Anhydrous citric acid | 5 | mg |
| 6. | L-Cysteine | 5 | mg |
| 7. | Lactose | 200 | mg |
| 8. | Mannitol | 547.5 | mg |
| 9. | Methylcellulose | 40 | mg |
| 10. | Glyceryl palmitostearate | 50 | mg |
| 11. | Berry flavours | 40 | mg |
| 12. | Potassium acesulfame | 0.5 | mg |
| 13. | Talc | 10 | mg |
| 14. | Sodium bicarbonate | 25 | mg |

### EXAMPLE IV

### 1000 mg Containing Chewable Tablets

| | | | |
|---|---|---|---|
| 1. | Vitis vinifera alcoholic extract | 80 | mg |
| 2. | Sanguinaria canadensis alcoholic extract | 2 | mg |
| 3. | Echinacea angustifolia lipophylic extract | 5 | mg |
| 4. | Soy lecithin | 30 | mg |
| 5. | Anhydrous citric acid | 5 | mg |
| 6. | L-Cysteine | 5 | mg |
| 7. | Lactose | 200 | mg |
| 8. | Mannitol | 507.5 | mg |
| 9. | Methylcellulose | 40 | mg |
| 10. | Glycerol palmitostearate | 50 | mg |
| 11. | Berry flavours | 40 | mg |
| 12. | Potassium acesulfame | 0.5 | mg |
| 13. | Talc | 10 | mg |
| 14. | Sodium bicarbonate | 25 | mg |

1. Compositions containing:
a. Anthocyanosides, and/or
b. Procyanidins,
c. Isoquinoline alkaloids extracted from Sanguinaria canadensis, Macleaya cordata or Macleaya microcarpa,
d. and optionally
a Zanthoxylum bungeanum and/or Echinacea angustifolia lipophilic extract.

2. Compositions as claimed in claim 1 wherein the anthocyanosides are extracted from Vaccinium myrtillus.

3. Compositions as claimed in claim 1 in which the procyanindins are extracted from Vitis vinifera.

4. Compositions as claimed in claim 1 in which the isoquinoline alkaloids are selected from sanguinarines and cheleritrines.

5. Compositions as claimed in claim 1 further containing a Zanthoxylum bungeanum and/or Echinacea angustifolia lipophilic extract.

6. Compositions as claimed in claim 1 in the form of tablets or chewing gums.

7. The use of:
a. Anthocyanosides, and/or
b. Procyanidins,
c. Isoquinoline alkaloids extracted from Sanguinaria canadensis, Macleaya cordata or Macleaya microcarpa,
and optionally
a. a Zanthoxylum bungeanum and/or Echinacea angustifolia lipophilic extract,
for the preparation of compositions for the prevention and the treatment of bacterial infections of the oral cavity.

8. The use of:
a. Anthocyanosides, and/or
b. Procyanidins,
c. Isoquinoline alkaloids extracted from Sanguinaria canadensis, Macleaya cordata or Macleaya microcarpa,
and optionally
d. a Zanthoxylum bungeanum and/or Echinacea angustifolia lipophilic extract, for the preparation of compositions for the hygiene of the oral cavity.
